(19) United States
(12) Patent Application Publication
(10) Pub. No.: US 2020/0129744 A1
(43) Pub. Date: Apr. 30, 2020

(54) PRE-MOISTENED SWAB SYSTEM

(71) Applicant: Inna Sych, Sacramento, CA (US)

(72) Inventor: Inna Sych, Sacramento, CA (US)

(21) Appl. No.: 16/663,802

(22) Filed: Oct. 25, 2019

Related U.S. Application Data
(60) Provisional application No. 62/750,727, filed on Oct. 25, 2018.

(51) Int. Cl. A61F 13/40; A61F 13/38; A61M 19/00; A61F 13/551; A45D 34/04

(52) U.S. Cl. CPC A61M 35/006; A61F 13/38; A61M 19/00; A45D 2200/1063; A45D 34/04; A61M 2209/06; A45D 2200/1036; A61F 13/551

(57) ABSTRACT

A pre-moistened swab system is disclosed including cotton swabs having a first end, a second end, swab tip, and a liquid material. The cotton swabs contained with the liquid material provide a pre-moistened swab applicator for use. The liquid materials may include makeup remover, rubbing alcohol or peroxide, anesthetic, petroleum jelly, and ear cleaner. It enables users to clean the face, moisturize dry lips, remove ear wax buildup, disinfect small wounds, numb the skin, and treat small cuts.

### CROSS REFERENCE TO RELATED APPLICATION

The present application is related to and claims priority to U.S. Provisional Patent Application No. 62/750,727 filed Oct. 25, 2018, which is incorporated by reference herein in its entirety.

### BACKGROUND OF THE INVENTION

The following includes information that may be useful in understanding the present disclosure. It is not an admission that any of the information provided is prior art nor material to the presently described or claimed inventions, nor that any publication or document that is specifically or implicitly referenced is prior art.

### 1. FIELD OF THE INVENTION

The present invention relates generally to the field of application devices and more specifically relates to cotton swabs.

### 2. DESCRIPTION OF RELATED ART

A dry cotton swab is not nearly as effective as a wet one; however, dry cotton swabs are what are available to the consumer. This forces the consumer to purchase a variety of solutions, such as ear cleaner, makeup remover and rubbing alcohol and moisten the tips of their cotton swabs. Manually moistening the swab is inconvenient and time consuming. The different solutions can also take up valuable storage space, especially when traveling. A suitable solution is desired.

U.S. Pat. No. 6,494,856 to Joseph Frank Zygmont relates to swab deliverable actives. The described swab deliverable actives includes a swab defined by an elongate stem and a fibrous absorbent covering such as cotton on at least one end of the stem. The fibrous absorbent covering is held together by a film-forming polymer. A treatment composition is dispersed onto the fibrous absorbent covering. Particularly preferred is a highly viscous hydrophobic treatment composition consisting of at least about 80% of petrolatum, which may serve to deliver actives such as bacitracin ointment or by itself serve as an occlusive therapeutic covering of skin to prevent infection.

### BRIEF SUMMARY OF THE INVENTION

In view of the foregoing disadvantages inherent in the known application devices art, the present disclosure provides a novel pre-moistened swab system. The general purpose of the present disclosure, which will be described subsequently in greater detail, is to provide users with cotton swabs that are pre-moistened with a specific solution.

A pre-moistened swab system is disclosed. The pre-moistened swab system includes a disposable swab applicator having a first end, a second end, a swab tip, and a medicated or non-medicated liquid material. The swab tip comprises a fibrous absorbent material such as but not limited to cotton. The swab tip contains the liquid material providing a pre-moistened swab applicator for use. The liquid material may be a liquid makeup remover, an ear cleaning solution, petroleum jelly, hydrogen peroxide, rubbing alcohol, an anesthetic solution, and an antibiotic ointment. The swab applicators may be provided in single or multi-use packs.

For purposes of summarizing the invention, certain aspects, advantages, and novel features of the invention have been described. It is to be understood that not necessarily all such advantages may be achieved in accordance with any one particular embodiment of the invention. Thus, the invention may be embodied or carried out in a manner that achieves or optimizes one advantage or group of advantages as taught without necessarily achieving other advantages as may be taught or suggested. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a pre-moistened swab system, constructed and operative according to the teachings of the present disclosure.

FIG. 1 is a perspective view of the pre-moistened swab system during an ‘in-use’ condition, according to an embodiment of the disclosure.

FIG. 2 is a perspective view of the pre-moistened swab system of FIG. 1, according to an embodiment of the present disclosure.

FIG. 3 is a perspective view of the pre-moistened swab system of FIG. 1, according to an embodiment of the present disclosure.

FIG. 4 is a perspective view of the pre-moistened swab system of FIG. 1, according to an embodiment of the present disclosure.

FIG. 5 is a perspective view of the pre-moistened swab system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to an application device and more particularly to a pre-moistened swab system as used to improve the application and general use of various solutions by providing a pre-moistened cotton swab device.

Generally, a pre-moistened swab system is a cotton swab with a tip pre-moistened in a variety of solutions. The invention is comprised of approximately 3″ long plastic rod with a cotton-covered tip. The tip is dipped into a solution. In some embodiments, the solutions include any one or any combination of a makeup remover, an ear cleaning solution, petroleum jelly, hydrogen peroxide or rubbing alcohol, an anesthetic solution, and an antibiotic ointment. The cotton swab packages are sealed to retain their moisture. It enables users to clean the face, moisturize dry lips, remove ear wax buildup, disinfect small wounds, numb the skin, and treat small cuts. The product affords convenience and ease in everyday life by providing pre-dipped swabs that can be used at home or taken on the go. The present invention eliminates the clutter associated with storing bottles of multiple solutions in a bathroom cabinet or luggage.

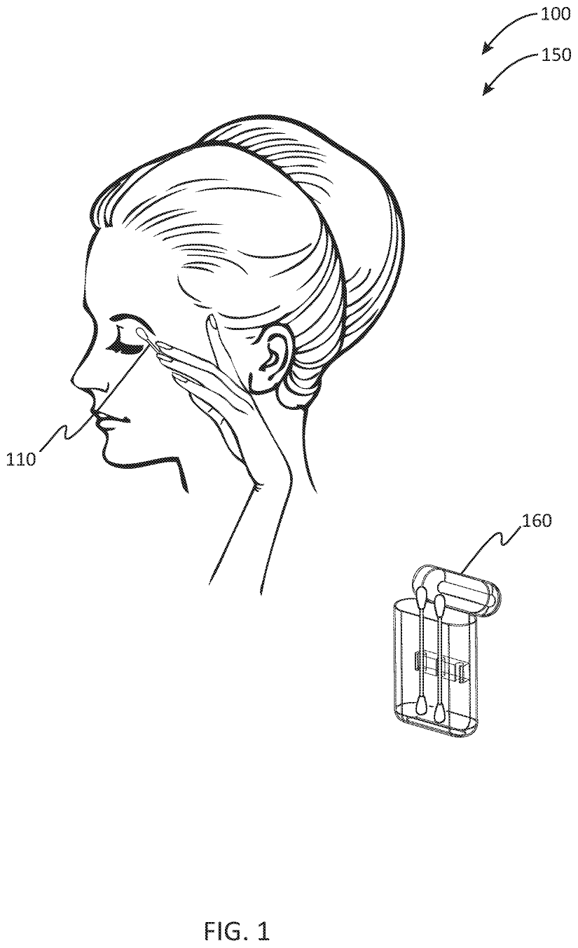

Referring now more specifically to the drawings by numerals of reference, there is shown in FIGS. 1-5, various views of a pre-moistened swab system 100. FIG. 1 shows a pre-moistened swab system 100 during an ‘in-use’ condition 150, according to an embodiment of the present disclosure. As illustrated, the pre-moistened swab system 100 may include a swab applicator 110 having a first end 112, a second end 116, a swab tip 120, and a liquid material 130. The swab tip 120 comprises a fibrous absorbent material. The swab tip 120 contains a liquid material 130 providing a pre-moistened swab applicator 110 for use.

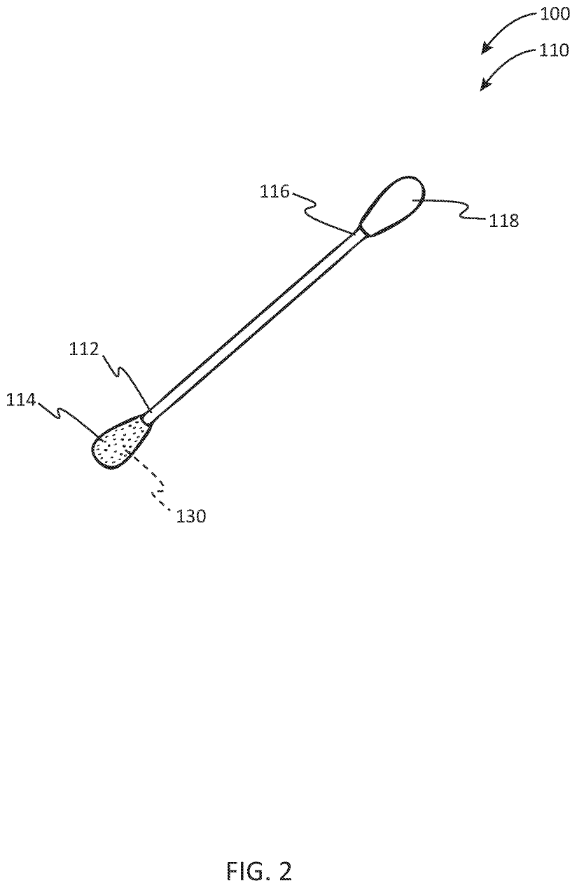

FIG. 2 shows a perspective view of the pre-moistened swab system 100 of FIG. 1, according to an embodiment of the present disclosure. As above, the pre-moistened swab system 100 may include the swab applicator 110 having the first end 112, the second end 116, swab tip 120, and the liquid material 130 providing a medicated or non-medicated cleaning or application device. The liquid material 130 may include but is not limited to a liquid makeup remover, an ear cleaning solution, petroleum jelly, hydrogen peroxide, rubbing alcohol, an anesthetic solution, and an antibiotic ointment.

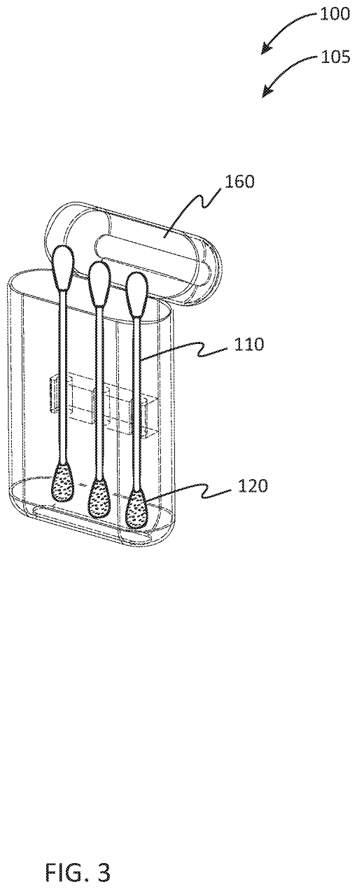

FIG. 3 shows a perspective view of the pre-moistened swab system 100 of FIG. 1, according to an embodiment of the present disclosure. As above, the pre-moistened swab system 100 may include the swab applicator 110 having the first end 112, the second end 116, swab tip 120 comprising the fibrous absorbent material such as cotton, and the liquid material 130. The swab tip 120 includes a first tip 114 and a second tip 118. The first tip 114 is positioned at the first end 112 of the swab applicator 110 and the second tip 118 is positioned at the second end 116 of the swab applicator 110. The first tip 114 includes the liquid material 130 and the opposing second tip 118 does not include the liquid material 130.

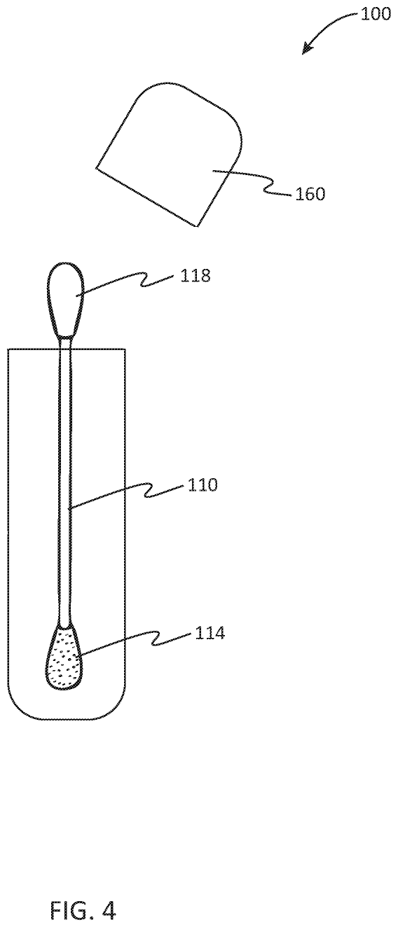

FIG. 4 shows a perspective view of the pre-moistened swab system 100 of FIG. 1, according to an embodiment of the present disclosure. As above, the pre-moistened swab system 100 may include the swab applicator 110 impregnated with the liquid material 130 such as a liquid makeup remover, an ear cleaning solution, petroleum jelly, hydrogen peroxide, rubbing alcohol, an anesthetic solution, and an antibiotic ointment. The swab applicator 110 is pre-moistened with the liquid material 130 and provided in a package 160. The package 160 may include multiple swab applicators 110 or a single individual swab applicator 110. For example, the hydrogen peroxide infused swab applicators 110 are provided in the package 160 including an individual disposable swab applicator 110. The swab applicator 110 may be plastic or non-plastic and is preferably disposable.

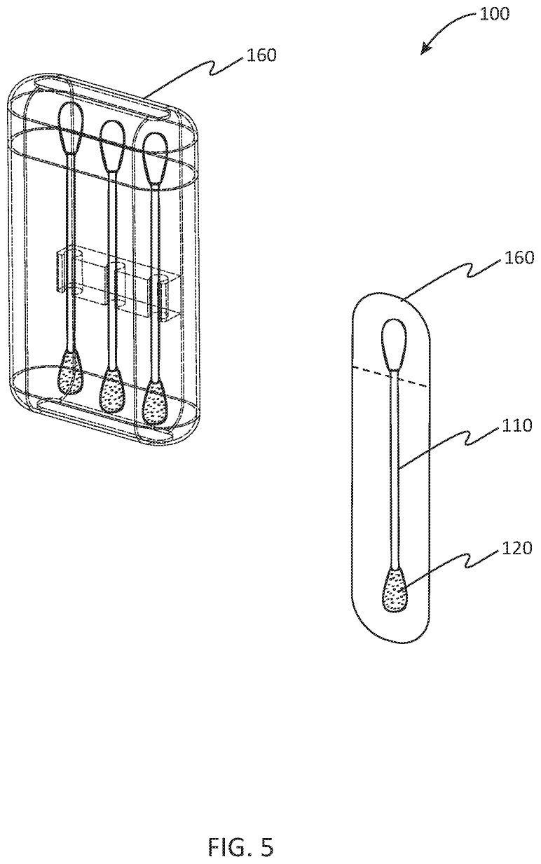

As shown in FIG. 5, according to one embodiment, the pre-moistened swab system 100 may be arranged as a kit 105. In particular, the pre-moistened swab system 100 may further include a set of instructions. The instructions may detail functional relationships in relation to the structure of the pre-moistened swab system 100 such that the pre-moistened swab system 100 can be used, maintained, or the like, in a preferred manner.

For purposes of this disclosure, “disposable” means that the device is intended and designed to only be used several times, such as 5, 4, 3, 2, or 1 time.

The embodiments of the invention described are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A pre-moistened swab system comprising:
a swab applicator having;
a first end;
a second end;
a liquid material; and
a swab tip comprising absorbent material;
wherein the swab tip is pre-moistened by the liquid material.

2. The pre-moistened swab system of claim 1, wherein the liquid material is any one or any combination of a liquid makeup remover, an ear cleaning solution, petroleum jelly, hydrogen peroxide, rubbing alcohol, an anesthetic solution, and an antibiotic ointment.

3. The pre-moistened swab system of claim 2, wherein the swab tip includes a first tip and a second tip.

4. The pre-moistened swab system of claim 3, wherein the first tip is positioned at the first end of the swab applicator and the second tip is positioned at the second end of the swab applicator.

5. The pre-moistened swab system of claim 4, wherein the first tip includes the liquid material.

6. The pre-moistened swab system of claim 5, wherein the second tip does not include the liquid material.

7. The pre-moistened swab system of claim 1, wherein the absorbent material of the swab tip comprises cotton.

8. The pre-moistened swab system of claim 1, wherein the swab applicator is plastic.

9. The pre-moistened swab system of claim 1, wherein the swab applicator is not plastic.

10. The pre-moistened swab system of claim 1, wherein the swab applicator is disposable.

11. The pre-moistened swab system of claim 1, wherein the swab applicator is pre-moistened with the liquid material and provided in a package.

12. The pre-moistened swab system of claim 11, wherein the package includes multiple the swab applicators.

13. The pre-moistened swab system of claim 11, wherein the package includes a single swab applicator.

14. The pre-moistened swab system of claim 13, wherein the swab applicator contains hydrogen peroxide and is provided individually in the package.

15. The pre-moistened swab system of claim 1, wherein the liquid material is medicated.

16. The pre-moistened swab system of claim 1, wherein the liquid material is non-medicated.

17. A pre-moistened swab system, the pre-moistened swab system comprising:
a swab applicator having;
a first end;
a second end;
a swab tip comprising cotton; and
a liquid material;
wherein the swab tip contains the liquid material;
wherein the liquid material is any one or any combination of a liquid makeup remover, an ear cleaning solution, petroleum jelly, hydrogen peroxide, rubbing alcohol, an anesthetic solution, and an antibiotic ointment;
wherein the first tip is positioned at the first end of the swab applicator and a second tip is positioned at the second end of the swab applicator;
wherein the first tip includes the liquid material;
wherein the swab applicator is non-plastic;
wherein the swab applicator is disposable; and
wherein the swab applicator is provided in a package.

18. The pre-moistened swab system of claim 17, further comprising a set of instructions; and
wherein the pre-moistened swab system is arranged as a kit.
